(19) United States
(12) Patent Application Publication
(10) Pub. No.: US 2023/0065180 A1
(43) Pub. Date: Mar. 2, 2023

(54) FLOW BLOCKING CATHETER

(71) Applicant: MICROPORT NEUROTECH (SHANGHAI) CO., LTD., Shanghai (CN)

(72) Inventors: Yuxi CUN, Shanghai (CN); Yunyun LIU, Shanghai (CN); Qinglong LIU, Shanghai (CN); Yumei LIU, Shanghai (CN)

(21) Appl. No.: 17/460,950

(22) Filed: Aug. 30, 2021

(51) Int. Cl. A61B 17/12

(52) U.S. Cl. CPC A61B 17/12109; A61B 17/1204; A61B 17/12136; A61B 17/12145; A61B 2017/22079

(57) ABSTRACT

A flow blocking catheter including an inner tube, an outer tube and a flow blocking member is provided. The outer tube is sleeved on an exterior of the inner tube and at least one end of the flow blocking member is coupled to the inner or outer tube. The ratio of the inner diameter of the inner tube to the outer diameter of the outer tube is equal to or greater than 0.7. With this arrangement, the inner diameter of the flow blocking catheter is able to be increased at a given outer diameter thereof, which makes it suitable for the treatment of large-size blood clots or passage of instruments and broadens its application range as well as improving effect of vascular treatments.

### TECHNICAL FIELD

The present application relates to the field of medical instruments and, in particular, to a flow blocking catheter.

### BACKGROUND

Strokes, mainly caused by blood clots in cerebral blood vessels, are a common medical condition that seriously threatens human health, which is also the third leading cause of death worldwide and the number one cause of long-term disability in adults. In the current clinical practice, treatments of directly sucking the thrombus with a suction catheter or removing the thrombus with the assistance of a stent are used to eliminate the thrombus to achieve recanalization of the blood vessel. After the suction catheter reaches the thrombus sit along the blood vessel, a negative pressure is applied at its proximal end to suck the clot into the suction catheter or onto the opening of suction catheter, followed by slow retraction of the clot into a guide catheter. As a result, the blood vessel recovers back to its normal hemodynamic condition. The stent-assisted thrombus remover is required to cross over the clot, trap the clot within meshes of the stent and then retract back into the support catheter, so as to recanalize the blood vessel. After the stent is retracted back into the support catheter, the support catheter together with the stent and blood clot trapped in the stent, is in turn withdrawn into the guide catheter. However, during the thrombus removal process, the clots often fall off and flow towards the distal blood vessel due to the impact of proximal blood flow, or during the operation of the suction catheter or the delivery of thrombus removal stent into the interventional instrument (the guideor support catheter) after the successful capture of clots, the clots break up and flow to distal end of the blood vessel to cause secondary blocking, which results in the failure of operation and may even threaten the patient's life in severe cases. For example, the possibility of percutaneous coronary intervention (PCI) caused myocardial necrosis reaches as high as 16%-39%, and most of these cases have been found to be attributable to escape of clots into distal blood vessels during the intervention operation. In order to solve the problems caused by clot fragmentation, the balloon guide catheter has been adpoted commonly in prior art to facilitate the thrombus removal operation by temporarily occluding the blood flow.

The flow blocking catheter refers to a type of catheter that is able to block the blood flow in the blood vessel with a certain manner. In existing flow blocking catheters, it is impossible to meet reqiurements of both inner diameter and outer diameter. In order to ensure the smooth passage of the catheter in blood vessels, the flow blocking catheter has to possess a small outer diameter. However, the inner diameter of the catheter is required to be as large as possible since the lumen of the flow blocking catheter is generally required to enable passage of instruments, such as support catheter, aspiration catheter or a stent, or to enable the accommodation of the captured blood clot. Generally, in order to ensure the smooth passage of the catheter in blood vessels, the outer diameter of the catheter must be designed to be not too large to result in a very small inner diameter, making it impossible to be fitted with a wide-lumen aspiration or support catheter. This therefore makes it unable to treat large-size thrombi. All these shortcomings limit the help function of existing flow blocking catheter in thrombus removal procedures, increase difficulty of such procedures and expose the patients to high risk.

### SUMMARY

It is an object of the present application to provide a flow blocking catheter to overcome the problems of impossibility to juggle size requirements of inner and outer diameter, small lumen and impossibility to fit with a large-size instrument or to treat a large-size thrombi that are present in existing guide catheters.

To solve the above problem, present application provides a flow blocking catheter comprising:

an inner tube;

an outer tube sleeved on the exterior of the inner tube; and

a flow blocking member having at least one end coupled to an outer circumference of the inner tube or the outer tube, the flow blocking member configured to have a collasped configuration and an expanded configuration,

wherein a ratio of an inner diameter of the inner tube to an outer diameter of the outer tube is equal to or greater than 0.7.

Optionally, the flow blocking member is configured to switch between the expanded and collapsed configurations under a drive of an axial movement of the outer tube relative to the inner tube.

Optionally, the flow blocking member is self-expandable and sleeved on an exterior of the inner tube. At least a proximal end of the flow blocking member is fixedly coupled to an outer circumference of the inner tube, and the outer tube is movably sleeved on an exterior of the inner tube to restrict expansion of the flow blocking member.

Optionally, one end of the flow blocking member is coupled to the outer circumference of the inner tube and the other end of the flow blocking member is coupled to an distal end of the outer tube. The flow blocking member is configured to expand as the outer tube moves toward a distal end of the inner tube and to collapse as the outer tube moves away from the distal end of the inner tube.

Optionally, the ratio of the inner diameter of the inner tube to the outer diameter of the outer tube is equal to or greater than 0.81.

Optionally, the ratio of the inner diameter of the inner tube to the outer diameter of the outer tube is equal to or greater than 0.85.

Optionally, the inner diameter of the inner tube ranges from 0.059 inch to 0.118 inch.

Optionally, the outer diameter of the outer tube ranges from 0.078 inch to 0.137 inch.

Optionally, both or either of the inner tube and the outer tube is a single-layered tube made of macromolecular material. Or, both or either of the inner tube and the outer tube has a structure comprising at least two layers, in which both or either of a first layer and a second layer from inside to outside is a macromolecular layer. Or, both or either of the inner tube and the outer tube has a structure comprising at least two layers, in which a second layer from inside to outside comprises one or more selected from the group consisting of braided structure, coil, and cut hypotube.

Optionally, the flow blocking member comprises at least one selected from the group consisting of mesh structure, open-loop structure, spiral structure and a balloon, and the flow blocking member is fabricated by braiding, winding, cutting, blow molding or extrusion.

Optionally, the mesh structure is braided from 1 to 64 filaments, wherein the filament is at least one selected from the group consisting of regular filament, radiopaque filament and composite filament, the regular filament made of at least one selected from the group consisting of nickel-titanium alloy, cobalt-chromium alloy, stainless steel and macromolecular material, the radiopaque filament made of at least one selected from the group consisting of radiopaque metal, alloy of radiopaque metals and macromolecular material containing a radiopaque agent, the composite filament formed by a radiopaque core filament combined with a regular filament.

In summary, the flow blocking catheter of the present application comprises an inner tube, a flow blocking member and an outer tube sleeved on the exterior of the inner tube, at least one end of the flow blocking member coupled to an outer circumference of the inner tube or the outer tube, the flow blocking member configured to have a collasped configuration and an expanded configuration. The ratio of an inner diameter of the inner tube to an outer diameter of the outer tube is equal to or greater than 0.7. With this configuration, the inner diameter of the flow blocking catheter is able to be increased at a given outer diameter thereof which makes it suitable for the treatment of large-size blood clots or instruments and broadens its application range as well as improving effect of vascular treatments.

### BRIEF DESCRIPTION OF THE DRAWINGS

Those of ordinary skill in the art would appreciate that the appended figures are presented merely to enable a better understanding of the present application rather than limit the scope thereof in any sense. In the figures,

FIG. 1 is a schematic diagram of a flow blocking catheter according to embodiment 1 of present application;

FIG. 2 is a schematic cross-sectional view of an inner tube according to embodiment 1 of present application;

FIG. 3 is a schematic diagram of an expanded flow blocking member according to embodiment 1 of present application;

FIG. 4 is a schematic diagram of a control valve according to embodiment 1 of present application;

FIG. 5 is a schematic cross-sectional view of a flow blocking catheter according to embodiment 1 of present application;

FIG. 6 is a schematic diagram of a flow blocking catheter provided with an groove according to embodiment 1 of present application;

FIG. 7 is a schematic diagram of a flow blocking catheter provided with a securing film according to embodiment 1 of present application;

FIG. 8 is a schematic diagram of a flow blocking member having its two ends fixedly secured to the inner tube according to embodiment 1 of present application;

FIG. 9 is a schematic diagram of a braided structure of a flow blocking member according to embodiment 1 of the present application; and

FIGS. 10a to 10g schematically illustrate various mesh openings of support frames according to preferred embodiments of the present application.

FIG. 11 is a schematic diagram of a flow blocking member in a collasped configuration according to embodiment 2 of present application;

FIG. 12 is a schematic diagram of a flow blocking member in an expanded configuration according to embodiment 2 of present application;

FIG. 13 is a schematic diagram of a flow blocking catheter provided with an expanded section according to embodiment 2 of present application;

FIG. 14 is a schematic diagram of a flow blocking catheter provided with an isodiametric section according to embodiment 2 of present application;

FIG. 15 is a schematic diagram of a flow blocking catheter provided with an isodiametric section according to embodiment 4 of present application

In the figures,

100, inner tube; 101, first layer; 102, second layer; 103, third layer; 104, adhesive; 110, groove; 120, first radiopaque ring; 130, expanded section;

200, outer tube; 210, distal end of the outer tube; 220, stress dispersion tube; 230, fluid introduction chamber; 231, fluid introduction hole;

300, flow blocking member; 310, first end; 320, second end; 330, radiopaque filament; 340, mesh opening; 350, balloon;

400, control valve; 410, control valve body; 420, control slider; 430, sliding slot; 440, catheter insertion opening; 500, securing film.

### DETAILED DESCRIPTION

To make objects, advantages and features of the present application more apparent, present application is described in detail by the particular embodiments made in conjunction with the accompanying drawings. Note that the figures are provided in a very simplified form not necessarily drawn to exact scale, with the only intention to facilitate convenience and clarity in explaining the present application. In addition, structures shown in the figures are usually part of actual structures. In particular, as the figures tend to have distinct emphases, they are often drawn to different scales.

As used in present specification, the meaning of “a,” “an,” and “the” include singular and plural references, unless the context clearly dictates otherwise. As used in present specification and appended claims, the term “or” genreally includes the meaning of “and/or”, unless the context clearly dictates otherwise. Additionally, the terms “proximal” and “distal” are generally used to refer to an end close to an operator and an end close to a lesion site in a patient, respectively. Further, the terms “one end” and “the other end”, or “proximal end” and “distal end”, are generally used to refer to two opposing portions including not only the endpoints.

The core idea of the present application is to provide a flow blocking catheter to overcome the problems of impossibility to juggle size requirements of inner and outer diameter, small lumen and impossibility to fit with a large-size instrument or to treat a large-size thrombi that are present in existing guide catheters. The flow blocking catheter comprises an inner tube, a flow blocking member and an outer tube. The outer tube is sleeved on an exterior of the inner tube and at least one end of the flow blocking member is coupled to the inner or outer tube. The flow blocking member is configured to have a collapsed configuration and an expanded configuration. The ratio of the inner diameter of the inner tube to the outer diameter of the outer tube is equal to or greater than 0.7. With this arrangement, the inner diameter of the flow blocking catheter is able to be increased at a given outer diameter thereof, which makes it suitable for the treatment of large-size blood clots or instruments and broadens its application range as well as improving effect of vascular treatments.

In the following description, reference is made to accompanying figures.

### Embodiment 1

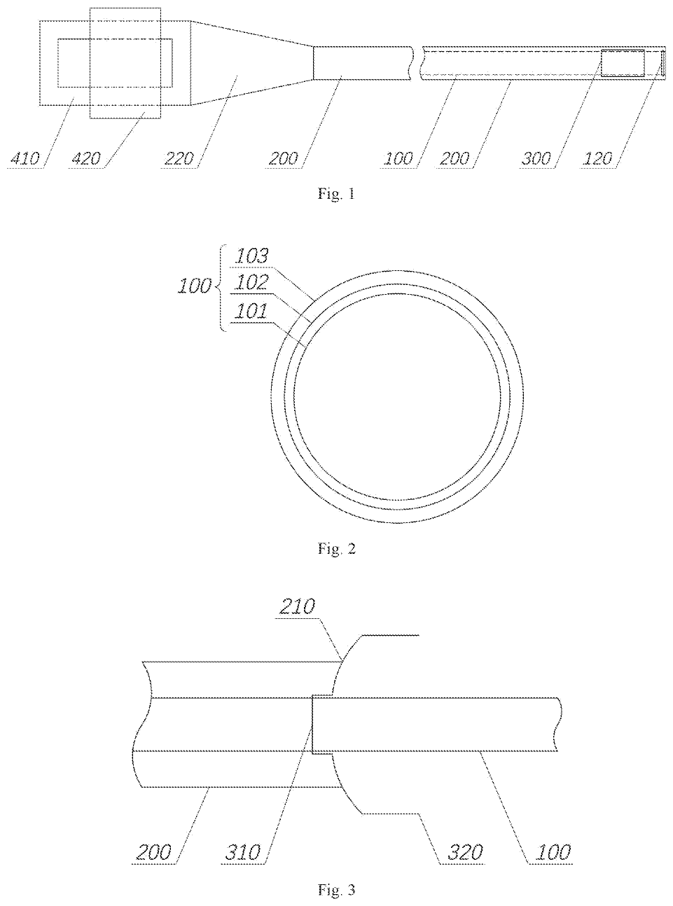

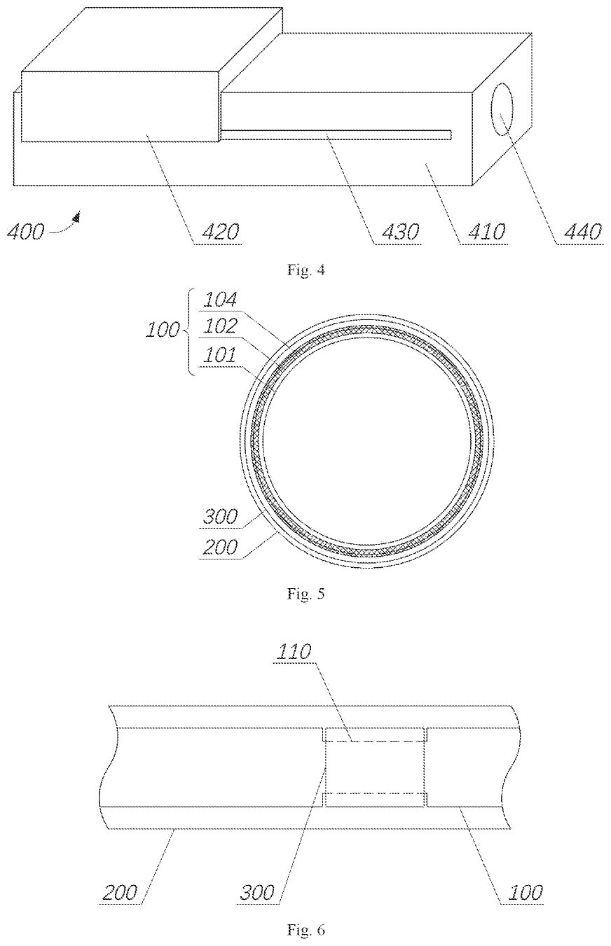

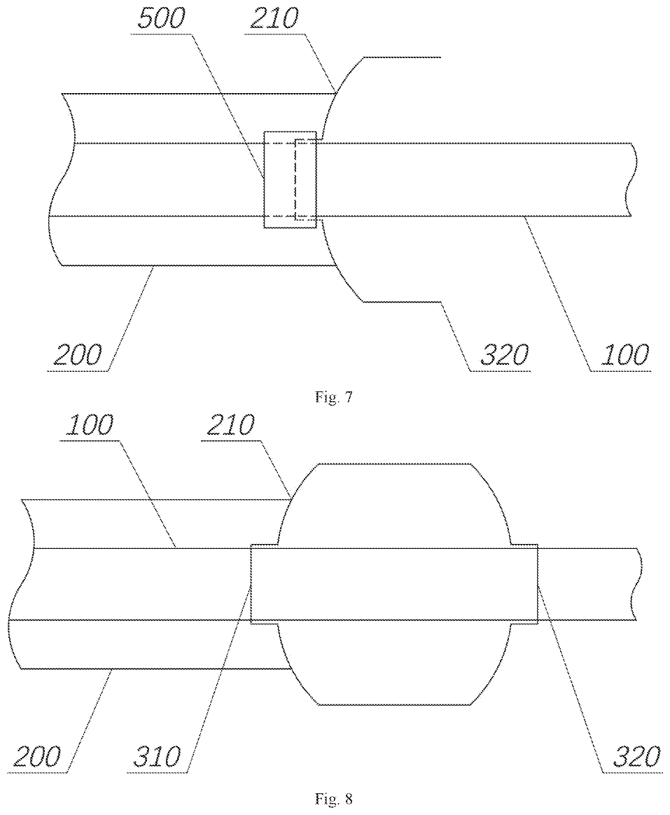

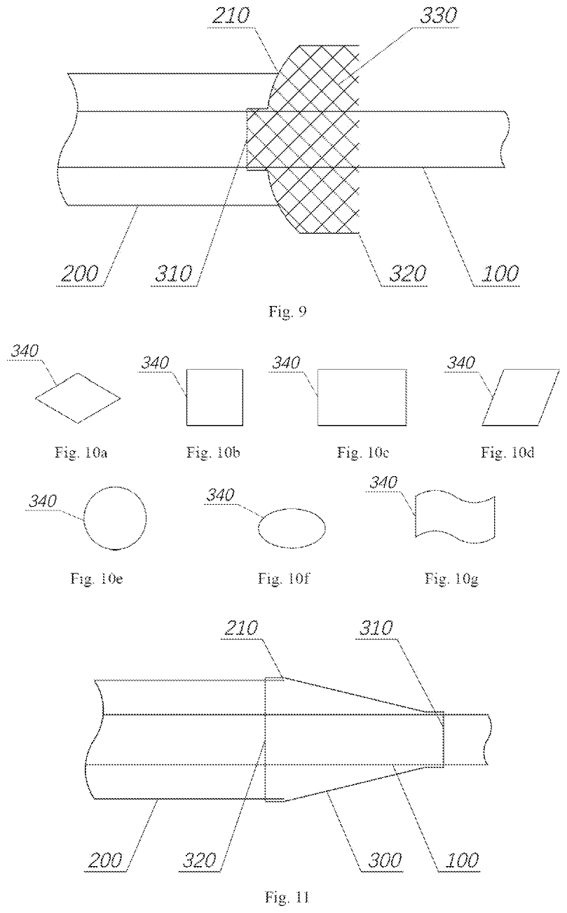

Please referring to FIG. 1 to FIG. 10g, in which FIG. 1 is a schematic diagram of a flow blocking catheter according to embodiment 1 of present application; FIG. 2 is a schematic cross-sectional view of an inner tube according to embodiment 1 of present application; FIG. 3 is a schematic diagram of an expanded flow blocking member according to embodiment 1 of present application; FIG. 4 is a schematic diagram of a control valve according to embodiment 1 of present application; FIG. 5 is a schematic cross-sectional view of a flow blocking catheter according to embodiment 1 of present application; FIG. 6 is a schematic diagram of a flow blocking catheter provided with a groove according to embodiment 1 of present application; FIG. 7 is a schematic diagram of a flow blocking catheter provided with a securing film according to embodiment 1 of present application; FIG. 8 is a schematic diagram of a flow blocking member having its two ends fixedly coupled to the inner tube according to embodiment 1 of present application; FIG. 9 is a schematic diagram of a braided structure of a flow blocking member according to embodiment 1 of present application; FIGS. 10a to 10g schematically illustrate various mesh openings of support frames according to preferred embodiments of the present application.

As shown in FIGS. 1 and 2, a flow blocking catheter according to an embodiment includes an inner tube 100, a flow blocking member 300 and an outer tube 200. The flow blocking member 300 is self-expandable. The flow blocking member 300 is sleeved on the exterior of the inner tube 100. At least proximal end(the first end 310) of the flow blocking member 300 is attached (e.g., by adhesive bonding, welding or a securing film) to an outer circumference of the inner tube 100. The outer tube 200 is movably sleeved on the exterior of the inner tube 100 in order to restrict expansion of the flow blocking member 300. The ratio of the inner diameter of the inner tube 100 to the outer diameter of the outer tube 200 is equal to or greater than 0.7. In some embodiments, the flow blocking member 300 is configured as that the outer tube 200 moves toward a proximal end of the inner tube 100 to remove its restriction to the flow blocking member 300, so that the flow blocking member 300 expands (radially) due to its own expandability; the outer tube 200 moves toward a distal end of the inner tube 100 to restrict expansion of the flow blocking member 300, so that the flow blocking member 300 contracts(and recovers along a radial direction). In another embodiments, the expansion and contraction of the flow blocking member 300 may be controlled by movements of the inner tube 100 relative to the outer tube 200. In this embodiment, the flow blocking member 300 has a first end 310 arranged close to the distal end of the inner tube 100 so that the flow blocking occurs at a location close to where a thrombus removal or other instrument operates, thus reducing adverse impact on blood flow around the proximal end. In alternative embodiments, the first end 310 of the flow blocking member 300 may be arranged at the middle or proximal end of the inner tube 100.

In one exemplary embodiment, both the inner 100 and outer 200 tubes are preferred to be circular tubes and the outer tube 200 is sleeved on the inner tube 100. The difference between an outer diameter of the inner tube 100 and an inner diameter of the outer tube 200 may range from 0.0001 inch to 0.1 inch. The outer tube 200 is preferred to be a single-layered tubular member formed of, for example, one or more of a polyether-polyamide block copolymer(PEBA or Pebax), polyamide (PA) and polytetrafluoroethylene (PTFE). The inner tube 100 includes at least a single macromolecular layer made of a macromolecular material that may be one or more selected from the group consisting of PTFE, high-density polyethylene (HDPE), Pebax mixed with a friction coefficient reducing additive, and polyolefin elastomer (POE). Preferably, the inner tube 100 includes a triple-layered structure, as shown in FIG. 3, consisting of a first layer 101, a second layer 102, and a third layer 103 arranged from inside to outside. The third layer 103 may be formed of for example, one or more of the nylon elastomer (e.g., Pebax), nylon and polyurethane (PU). The first layer 101 may be formed of for example, one or more of PTFE, HDPE, Pebax mixed with a friction coefficient reducing additive, and POE. The second layer 102 may consist of any one of a braided structure, a coil and a cut hypotube (here, the term “hypotube” refers to any metal tube for medical use), or a combination of two or more thereof. The second layer 102 may be formed of stainless steel, nickel-titanium alloy, cobalt-chromium alloy or macromolecular wire, which can increase force transmission performance, ellipticity resistance and bending resistance of the inner tube 100 as well as reduce a force required to withdraw the flow blocking member 300. It is to be understood that materials of the layers of the inner tube 100 are not limited to the materials listed above, and those skilled in the art may also choose other functionally similar materials based on prior art. As shown in FIG. 5, in one alternative embodiment, the inner tube 100 includes only two layers, which are a first layer 101 and a second layer 102 covered on the first layer. In this case, the first layer 101 may be essentially a macromolecular layer formed of one or more of PTFE, HDPE, Pebax mixed with a friction coefficient reducing additive, and POE. The second layer 102 may be essentially a metallic layer consisting of, for example, any one of a braided structure, a coil and a cut hypotube, or a combination of two or more thereof. The second layer 102 may be formed of, for example, stainless steel, a nickel-titanium alloy, a cobalt-chromium alloy or the like. Preferably, a layer of adhesive 104 may be applied onto the macromolecular layer, which penetrates into the metallic layer (i.e., part of the adhesive 104 penetrates through meshes formed in the metallic layer and adheres to the exterior of the macromolecular layer) to form a firm adhesion between the metallic and macromolecular layers, so as to improve the force transmission performance and ellipticity resistance. Of course, the outer tube 200 is not limited to being a single-layered tube in other embodiments, and it may also be implemented as a double-, triple- or more-layered structure. The specific structure of the outer tube 200 can refer to that of the inner tube 100.

Preferably, the inner tube 100 includes a first radiopaque ring 120 disposed at the distal end of the inner tube 100. In particular, the first radiopaque ring 120 may be disposed at a distal end of the second layer 102 in the inner tube 100. More preferably, the inner tube 100 further includes a second radiopaque ring (not shown)disposed at a location of the inner tube 100 where the flow blocking member 300 is attached to the inner tube 100. Further, when one end of the flow blocking member 300 is attached to the outer circumference of the inner tube 100 and the other end of the flow blocking member 300 is a free end, the flow blocking member 300 further includes a third radiopaque ring (not shown) disposed at the free end thereof. The design of the third radiopaque ring allows to visually reflect to what extent the free end of the flow blocking member 300 expands. Optionally, examples of materials of the first, second and third radiopaque rings may include, but are not limited to, platinum, iridium, tantalum, noble metal alloys and macromolecular materials containing radiopaque agents. Arranging the three radiopaque rings helps the operator locate the inner tube 100 during a surgical procedure, or enables visual reflection of expansion extent of the flow blocking member 300. It is to be understood that the first radiopaque ring 120 is located at the distal end of the inner tube 100, but it is not intended to limit that the first radiopaque ring 120 can only be located at the distal end face of the inner tube 100, which can be located in an area close to the distal end of the inner tube 100. While the above embodiment exemplifies the positions of the three radiopaque rings, it is not intended to limit that the three radiopaque rings must be provided simultaneously, and those skilled in the art may select to provide any one or two of them according to the actual circumstances.

Preferably, the flow blocking member 300 includes a support frame, which is attached at least at its proximal end to the outer circumference of the inner tube 100 and is self-expandable. Optionally, the flow blocking member 300 may further include an flow blocking membrane attached to the support frame. In one example, the support frame is a tubular member that is able to switch between a collapsed configuration and an expanded configuration under the restriction of the outer tube 200. It is to be understood that the support frame is not limited to switch only between the collapsed configuration and the expanded configuration. In some cases, it may also assume an intermediate configuration between the collapsed and expanded configurations (i.e., a semi-expanded or partially-expanded configuration). The support frame may be formed of for example, nickel-titanium alloy, Type 304 stainless steel, platinum-tungsten alloy, platinum-iridium alloy, cobalt-chromium alloy, radiopaque metal or the like. The support frame may be fabricated by winding, cutting or braiding.

In this embodiment, the support frame includes a plurality of mesh openings 340, as shown in FIGS. 10a to 10g. The mesh opening 340 may have a rhombic(10a), square(10b), rectangular(10c), parallelogramic(10d), polygonal(not shown), circular (10e), elliptic(10f) or irregular (10g) shape, with the rhombic shape (10a) being preferred. The flow blocking membrane may be attached to either an inner surface or an outer surface of the support frame. The flow blocking membrane is preferably a macromolecular membrane formed of, for example, PU, polyethylene(PE), expanded polytetrafluoroethylene (ePTFE) or the like. It is to be understood that the support frame and the flow blocking membrane are not limited to being formed of the materials listed above, and those skilled in the art may also choose other functionally similar materials based on prior art. As shown in FIG. 9, in some embodiments, the support frame may be a mesh structure braided from 1-64 filaments. The filament may be at least one selected from the group consisting of regular filament, radiopaque filament and composite filament. The material of the regular filament may be selected as, but is not limited to, one or more of nickel-titanium alloy, cobalt-nickel alloy, stainless steel, macromolecular material and the like. The material of the radiopaque filament may be selected as, but is not limited to, radiopaque metal such as platinum, iridium, gold or tungsten, or the alloy thereof, or a macromolecular material containing a radiopaque agent of the radiopaque metal or alloy. The composite filament may be a bilayer structure consisting of a radiopaque core filament and a regular filament coated on the radiopaque core filament. The inner radiopaque core filament may be formed of one or more of a radiopaque metal such as platinum, iridium, gold or tungsten, or an alloy thereof, while the outer regular filament may be formed of one or more of the nickel-titanium alloy, cobalt-nickel alloy, stainless steel and macromolecular material. The radiopaque filament 330 imparts an improved radiographic visibility to the flow blocking member 300, and thus imparts an improved traceability to the flow blocking member 300 during use. In alternative embodiments, the support frame may be an open-loop structure or a spiral structure, or consist of two or more of a mesh structure, an open-loop structure and a spiral structure.

Referring to FIGS. 1 and 3, expansion state of the flow blocking member 300 (it would be appreciated that expansion state of the flow blocking member 300 is the same as that of the support frame) can be controlled by movement of the outer tube 200 along an axis of the inner tube 100. Specifically, in an initial configuration of the flow blocking catheter, the distal end 210 of the outer tube compresses the flow blocking member 300 to restrict expansion of the flow blocking member 300, where the flow blocking member 300 is in a collapsed configuration. Moreover, in this configuration, the flow blocking member 300 is compressed between the inner 100 and outer 200 tubes, facilitating movement of the flow blocking catheter in a blood vessel. When the outer tube 200 moves towards the proximal end relative to the inner tube 100(i.e., the outer tube 200 is retracted), the flow blocking member 300 is exposed from the outer tube 200, and self-expands to press against the wall of a blood vessel. The blood flow in the blood vessel is thus blocked as a layer of the flow blocking membrane is attached to the support frame of the flow blocking member 300. It is to be understood that, at this point of time, the expansion of the flow blocking member 300 adapts to the dimension of the blood vessel wall, and the flow blocking member 300 is not necessarily fully expanded (i.e., flow blocking member 300 may be in a semi-expanded configuration). Of course, in some other cases, when the outer tube 200 retracts, the distal end 210 of the outer tube 200 may not separate from the flow blocking member 300 and only a part of the flow blocking member 300 is no longer restricted by the outer tube 200. Such part of the flow blocking member 300 thus self-expands to press against the blood vessel wall to block the blood flow. That is, the outer tube 200 may release and allow expansion of only part of the flow blocking member 300, but does not necessarily release and allow expansion of the entire flow blocking member 300 (i.e., the flow blocking member 300 may be in a partially-expanded configuration). Preferably, the flow blocking member 300 is compliant to a certain extent, which makes it able to adapt shapes of the blood vessel walls in an expanded configuration (including the fully-, semi- or partially-expanded configuration). This arrangement is favorable to blood vessels with vulnerable walls by reducing the force applied to these blood vessel walls from the expansion of the flow blocking member 300. As a result, the flow blocking member 300 is able to lower stimulation to the wall of a cerebral blood vessel, suppress the occurrence of various complications such asvasospasm during surgery and completely avoid the risk of secondary damage to the blood vessel caused by the bursting of a balloon or a balloon bonding section.

Further, when blood flow blocking has been attained, a blood clot can be directly sucked, or captured and pulled back via the lumen of the inner tube 100(a aspiration catheter may be deployed in the lumen of the inner tube 100 of the flow blocking catheter to suck the clot, or a support catheter may be deployed in the lumen, in which a thrombectomy stent is provided for removing the clot). As shown in FIG. 2, because of a relative small thickness of the flow blocking member 300 in the collapsed configuration, the lumen of the inner tube 100 takes up a much larger proportion of a cross-sectional area of the flow blocking catheter, when compared to the case of using a conventional balloon catheter. Therefore, for a given outer diameter, the flow blocking catheter of present application is able to be fitted with aspiration catheters or support catheters with large lumens, larger size stents or other medical instruments for treating large blood clots, while the outer diameter of the entire flow blocking catheter is limited to ensure that the flow blocking catheter is able to pass through tortuous distal blood vessels successfully and causes a small wound to the patient.

Further, when it is necessary to change positions of the blood flow blocking by relocating or withdrawing the flow blocking catheter, the outer tube 200 may be caused to move distally relative to the inner tube 100 (i.e., retracting the outer tube 200) until the distal end of the outer tube 200 comes into abutment against the flow blocking member 300, as shown in FIG. 3. Then, the outer tube 200 may be continuously pushed towards the distal end until the flow blocking member 300 is compressed into the collapsed configuration. The configuration of the flow blocking member 300 shown in FIG. 3 is reversible. That is, the flow blocking member 300 self-expands again, when the outer tube 200 is retracted proximally. The ability of the flow blocking member 300 to be collapsed repeatedly enables easy re-delivery and relocation of the flow blocking catheter. Therefore, the flow blocking catheter of this embodiment provides convenience for achieving repetitive operations, accurate location as well as convenience for achieving withdrawal from the blood vessel with the removed thrombus.

As shown in FIG. 4, the flow blocking catheter further includes a control valve 400 configured to drive movements of the outer tube 200 relative to the inner tube 100. In one embodiment, the control valve 400 includes a control valve body 410 and a control slider 420. The control valve body 410 is provided with a sliding slot 430 along its axial direction. The sliding slot 430 matches with the control slider 420 to enable the control slider 420 to slide along the sliding slot 430. Further, one end of the control valve body 410 defines a catheter insertion opening 440, through which proximal end of the inner tube 100 inserts into the control valve 400 and fixedly coupled to the control valve body 410 while the proximal end of the outer tube 200 is coupled to the control slider 420, for example, by adhesive bonding or snap-fitting. With this arrangement, movements(e.g., backward retraction or forward push) of the outer tube 200 relative to the inner tube 100 is able to be controlled by controlling sliding of the control slider 420. In this way, the control valve 400 is able to control expansion or contraction of the flow blocking member 300, thus simplifying operations involved in the surgical procedure, shortening the operation time and providing convenience for repeat operations. Optionally, proximal end of the outer tube 200 includes a stress dispersion tube 220. The stress dispersion tube 220 flares towards the proximal end. That is, the distal end of the stress dispersion tube 220 has a diameter equal to the diameter of the outer tube 200, and the proximal end of the stress dispersion tube 220 has a diameter greater than the diameter of the outer tube 200. With this arrangement, portion of the outer tube 200 configured to couple the control slider 420 has a widened diameter, and the flaring stress dispersion tube 220 helps in dispersing a drive force exerted by the control slider 420 on the outer tube 200, enabling to achieve a more reliable control of outer tube 200 by the control slider 420. In alternative embodiments, it is also possible to couple the outer tube to the control valve body 410, with the inner tube 100 being coupled to the control slider 420. Other direct or indirect coupling designs are also possible, and the present application is not limited in this regard.

Referring to FIG. 6, in one preferred embodiment, a groove 110 is disposed along the outer circumference of the inner tube 100. The shape of the groove 110 matches with the shape of the flow blocking element 300 in the collapsed configuration so as to accommodate the flow blocking element 300. Optionally, the groove 110 is an annular groove surrounding the inner tube 100. When fully collapsed, the flow blocking member 300 can be embedded in the groove 110. Preferably, the groove 110 have a length that is equal to or greater than a length of the flow blocking member 300 in the collapsed configuration so that the flow blocking member 300 can be entirely accommodated in the groove 110. The groove 110 allows an even smaller gap between the outer tube 200 and the inner tube 100, thus additionally reducing the proportion of the cross-sectional area of the tube accounting for the cross-sectional area of the entire flow blocking catheter, and increasing the proportion of the cross-sectional area of inner lumen(i.e., the inner lumen of inner tube 100) accounting for the cross-sectional area of the entire flow blocking catheter. The groove 110 allows the flow blocking catheter to maintain a constant outer diameter throughout its whole length. This can avoid damage to the flow blocking member 300 when the flow blocking catheter is passing through a tortuous blood vessel. Moreover, the locations of the inner tube 100 where the flow blocking member 300 is attached to the inner tube 100 are spaced from the central axis of the inner tube 100 by substantially equal radial distances, which is helpful in maintaining concentricity, and avoiding eccentricity, of the flow blocking member 300 during its expansion. As a result, the uniformity of the attachment of flow blocking member 300 to the blood vessel wall is able to be achieved, so as to reduce the risk of blood leakage.

Referring to FIG. 3, in one preferred embodiment, one end of the flow blocking member 300 is attached to the outer circumference of the inner tube 100, for example, by adhesive bonding or welding, and the other end of the flow blocking member 300 is a free end. Optionally, the first end 310 (e.g., the proximal end) of the flow blocking member 300 is attached to an outer surface of the inner tube 100, with its second end 320 (e.g., the distal end) being a free end. In this case, in an expanded configuration of the flow blocking member 300, the second end 320 is spaced from the distal end of the inner tube 100 by a distance in the range of 0-500 mm. It is to be understood that the second end 320 should not extend out of the distal end of the inner tube 100. That is, the flow blocking member 300 is entirely located closer to the operator than the distal end of the inner tube 100.

As shown in FIG. 7, preferably, the flow blocking catheter further includes a securing film 500 attached to the exterior of the proximal end of the flow blocking member 300 so as to cover at least part of the flow blocking member 300 and part of the inner tube 100. The attachment of the securing film 500 to the flow blocking member 300 and the inner tube 100 may be accomplished by, for example, adhesive bonding or heat shrinkage. The securing film 500 allows to strengthen the fixation between the flow blocking member 300 and the inner tube 100. Optionally, the securing film 500 has an axial length ranging from 1 mm to 10 mm. It is to be noted that, in some embodiments, the flow blocking member 300 may be first attached to the inner tube 100 (e.g., by adhesive bonding or welding), and the securing film 500 may be then applied as a secondary reinforcing fixture for enhancing the reliability of the fixation. In other embodiments, the flow blocking member 300 may be dimensioned to have a dimensional fit or an interference fit with the inner tube 100 and is secured to the inner tube 100 by the securing film 500. However, the present application is not limited in this regard.

Referring to FIG. 8, in another preferred embodiment, the flow blocking member 300 is attached to the outer circumference of the inner tube 100 at two ends by, for example, adhesive bonding, welding or a securing film. Optionally, the two ends of the flow blocking member 300 are spaced apart by a certain distance. With this arrangement, when expanded, the flow blocking member 300 appears like a fusiform shape, which allows to achieve a better contact with the blood vessel wall and thus an improved blood flow blocking effect. In addition, the shape of the flow blocking element 300 is more stable under the impact of blood flow, additionally reducing the risk of blood leakage. Of course, in some other embodiments, it is also possible to arrange the two ends of the flow blocking member 300 adjacent to, or even overlapping with each other. In these cases, the flow blocking member 300 in the expanded configuration assumes a shape resembling the Greek letter Ω, while still providing similar benefits. Therefore, the present application is not limited the distance between two ends of the flow blocking member 300.

### Embodiment 2

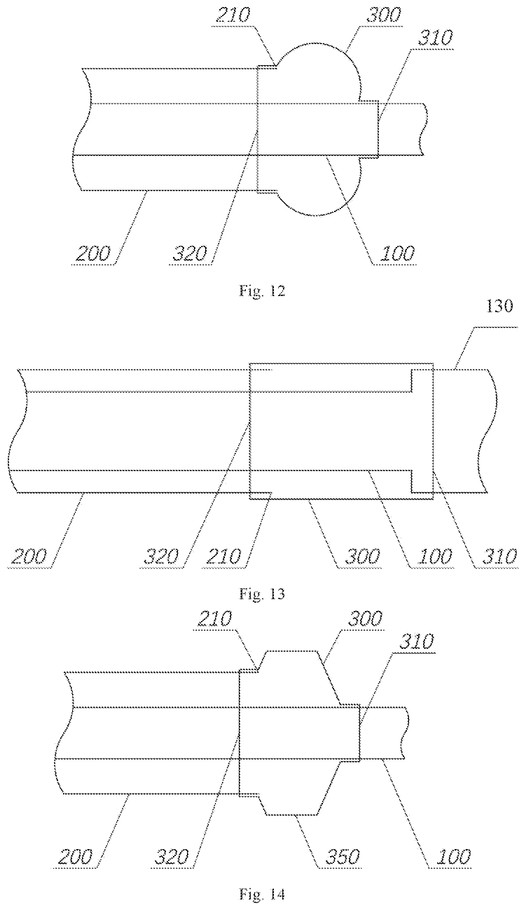

Please referring to FIG. 11 to FIG. 14, in which FIG. 11 is a schematic diagram of a flow blocking member in a collasped configuration according to embodiment 2 of present application; FIG. 12 is a schematic diagram of a flow blocking member in an expanded configuration according to embodiment 2 of present application; FIG. 13 is a schematic diagram of a flow blocking catheter provided with an expanded section according to embodiment 2 of present application; FIG. 14 is a schematic diagram of a flow blocking catheter provided with an isodiametric section according to embodiment 2 of present application.

The flow blocking catheter of Embodiment 2 is substantially similar to that of Embodiment 1, and only differences between these flow blocking catheters are described below, with the similarities between them being omitted.

In Embodiment 2, the flow blocking memer is arranged different from the flow blocking memer of Embodiment 1. Specifically, referring to FIGS. 11-12, in this Embodiment, one end(the first end 310) of the flow blocking member 300 is attached (e.g., by adhesive bonding, welding or a securing film) to an outer circumference of the inner tube 100 and the other end (the second end 320) of the flow blocking member 300 is attached (e.g., by adhesive bonding, welding or a securing film) to the distal end of the outer tube 200(distal end 210 of the outer tube). The flow blocking member 300 is configured to expand (i.e., bulge radially) as the outer tube 200 moves toward a distal end of the inner tube 100 and to collapse (i.e., retract and recover radially) as the outer tube 200 moves away from the distal end of the inner tube 100(i.e., towards a proximal end of the inner tube 100). In still other embodiments, expansion and collapse of the flow blocking member 300 is able to be controlled by movements of the inner tube 100 relative to the outer tube 200. In this embodiment, the first end 310 of the flow blocking member 300 is arranged close to the distal end of the inner tube 100 so that blood blocking position is close to the location where a thrombus removal or other instrument operates, thus reducing adverse impact on blood flow in the proximal blood vessel. In alternative embodiments, the first end 310 of the flow blocking member 300 may also be arranged at the middle of the inner tube 100 or close to the proximal end of the inner tube 100.

Continuing to refer to FIGS. 11-12, expansion state of the flow blocking member 300 (it would be appreciated that expansion state of the flow blocking member 300 is the same as that of the support frame) can be controlled by movement of the outer tube 200 along an axis of the inner tube 100. Specifically, as shown in FIG. 11, for ease of description, the fixing point where the first end 310 of the flow blocking member 300 is fixed to the inner tube 100 is referred to hereinafter as a first fixing point, and the fixing point where the second end 320 of the flow blocking member 300 is fixed to the outer tube 200 as a second fixing point. In an initial configuration of the flow blocking catheter, distance between the first fixing point and the second fixing point along the axial direction of the inner tube 100 is the largest. At this moment, the flow blocking member 300 is in a fully collapsed configuration with a maximum outer diameter that is comparable to an outer diameter of the outer tube 200. Based on the configuration of FIG. 11, the outer tube 200 is pushed distally to decrease the axial distance between the first and second attachment points, which causes the flow blocking member 300 to expand outward along its radial direction, as shown in FIG. 12 where the flow blocking member 300 is in a fully expanded configuration. When the flow blocking member 300 expands outwardly to the extent that it fits the inner diameter of the blood vessel wall, the flow blocking member 300 contacts and attaches to the blood vessel wall. The blood flow in the blood vessel is thus blocked as a layer of the flow blocking membrane is attached to the support frame of the flow blocking member 300. It is to be understood that, at this point of time, the expansion of the flow blocking member 300 adapts to the dimension of the blood vessel wall, and the flow blocking member 300 is not necessarily fully expanded (i.e., flow blocking member 300 may be in a semi-expanded or patially-expanded configuration). Preferably, the flow blocking member 300 is compliant to a certain extent, which makes it able to adapt shapes of the blood vessel walls in an expanded configuration (including the fully-, semi- or partially-expanded configuration). This arrangement is favorable to blood vessels with vulnerable walls by reducing the force applied to these blood vessel walls from the expansion of the flow blocking member 300. As a result, the flow blocking member 300 is able to lower stimulation for the wall of a cerebral blood vessel, suppresses the occurrence of various complications such asvasospasm during surgery and completely avoids the risk of secondary damage to the blood vessel caused by the bursting of a balloon or a balloon bonding section.

Further, when it is necessary to change positions of the blood flow blocking by relocating or withdrawing the flow blocking catheter, the outer tube 200 may be caused to move proximally relative to the inner tube 100 (i.e., retracting the outer tube 200) until the distance between the first and second fixing points along the axial of the inner tube 100 becomes maximum. The configuration of the flow blocking member 300 shown in FIG. 12 is reversible when the outer tube 200 is retracted proximally, i.e., the flow blocking member 300 is able to be collapsed to return to the configuration shown in FIG. 11. The repeatable collapse of the flow blocking member 300 enables easy re-delivery and relocation of the flow blocking catheter. Therefore, the flow blocking catheter of this embodiment provides convenience for achieving repetitive operations, accurate location as well as convenience for achieving withdrawal from the blood vessel with the removed thrombus.

Referring to FIG. 13, in one preferred embodiment, the inner tube 100 includes a expanded section 130 at the outer circumference of its distal end. The expanded section 130 has an outer circumferential size greater than that of rest portions of the inner tube 100. In this case, the blood blocking member 300 is connected to the expanded section 130 at one end (the first end 310) and to the distal end 210 of the outer tube at the other end (the second end 320). Preferably, the outer circumferential size of the expanded section 130 is matched with that of the outer tube 200, and the expanded section 130 is located distally relative to the distal end of the outer tube 200. In one exemplary embodiment, the main section of the inner tube 100 has an outer diameter of 0.070-0.113 inches and a length of 70-100 mm, while the expanded section 130 has an outer diameter of 0.079-0.122 inches and a length of 1-50 mm. Preferably, an inner diameter of the outer tube 200 is slightly greater than the outer diameter of the main section of the inner tube 100, and the outer tube 200 is sleeved on the main section of the inner tube 100. The outer diameter of the outer tube 200 may be equal to that of the expanded section 130. Preferably, the axial distance between the distal end 210 of the outer tube and the expanded section 130 may be 5-50 mm. Moreover, the first fixing point between the first end 310 of the blood blocking member 300 and the expanded section 130 is close to an fixing point between the expanded section 130 and the main section of the inner tube 100; and the second fixing point between the second end 320 of the blood blocking member 300 and the outer tube 200 is close to the distal end 210 of the outer tube. The expanded section 130 of the inner tube 100 is designed to enable the blood blocking catheter to maintain a constant outer diameter throughout its whole length. This can avoid damages to the blood blocking member 300 when the blood blocking catheter is passing through a tortuous blood vessel during its delivery in the blood vessel. Moreover, radial distance of the first fixing point relative to the axis of the inner tube 100 substantially equals the distance of the second fixing point relative to the central axis of the inner tube 100, which is helpful in maintaining concentricity and avoiding eccentricity, of the blood blocking member 300 during its expansion as well as improving the uniformity of contaction of the blood blocking member 300 to the blood vessel wall to reduce the risk of blood leakage.

Referring to FIG. 14, in one preferred embodiment, the blood blocking member 300 includes an isodiametric section 350. When the blood blocking member 300 is in an expanded configuration, the isodiametric section 350 maintains a constant outer circumferential size along an axis thereof (i.e., the isodiametric section 350 has a cylindrical shape). Optionally, the expanded configuration of the isodiametric section 350 of the blood blocking member 300 is thermally formed to be a tube having a constant diameter. With this arrangement, when the blood blocking member 300 is axially compressed to expand, the isodiametric section 350 expands synchronously throughout its axial length. This results in a shape of the blood blocking member 300 that allows the blood blocking member 300 to better press against the blood vessel wall, which thus provides an even desirable blood blocking performance. It is to be understood that, in some embodiments, the isodiametric section 350 is not limited to expand synchronously throughout its length during the expansion of the blood blocking member 300 and is designed to expand gradually from one end to the other end. In this case, the isodiametric section 350 possesses a sloped outer surface at first(i.e., the isodiametric section 350 has a tapered shape as a whole), and then achieves the configuration having a constant outer circumferential size along its axial direction (i.e., the isodiametric section 350 has a cylindrical shape) until the blood blocking member 300 has expanded fully or to an extent adapted to the inner diameter of the blood vessel wall. However, the present invention is not limited in this regard.

### Embodiment 3

The flow blocking catheter of Embodiment 3 is substantially similar to that of Embodiment 1, and only differences between these flow blocking catheters are described below, with the similarities between them being omitted.

In the flow blocking catheter of Embodiment 3, the inner tube 100, outer tube 200 and the flow blocking member 300 have different specific dimensions and structures from Embodiment 1. Optionally, the ratio of the inner diameter of the inner tube 100 to the outer diameter of the outer tube 200 is equal to or greater than 0.76. Preferably, the ratio of the inner diameter of the inner tube 100 to the outer diameter of the outer tube 200 is equal to or greater than 0.81. More preferably, the ratio of the inner diameter of the inner tube 100 to the outer diameter of the outer tube 200 is equal to or greater than 0.85. Further, the inner diameter of the inner tube 100 is in the range of 0.059-0.118 inch, while the outer diameter of the outer tube 200 ranges from 0.078 inch to 0.137 inch. This will be explained in greater detail below by way of preferred examples.

In the first preferred example of Embodiment 3, the inner tube 100 includes a triple-layered structure including a first layer 101, a second layer 102 and a third layer 103 that are arranged from inside to outside. The first layer 101 has a thickness of 0.001 inch. The second layer 102 has a braided structure made from filaments with a diameter of 0.002 inch. The third layer 103 has a thickness of 0.003 inch. The outer tube 200 includes a single-layered structure with a thickness of 0.003 inch. The flow blocking member 300 is disposed between the inner tube 100 and the outer tube 200 and includes a support frame and a covering membrane. The support frame is a cut tube with a wall thickness of 0.004 inch. The covering membrane is disposed outside the support frame and has a thickness of 0.0025 inch. An outer diameter of the whole flow blocking catheter (i.e., the outer diameter of the outer tube 200) is 0.117 inch, and the inner diameter of the inner tube 100 is 0.082 inch. Therefore, the ratio of inner to outer diameter of the entire flow blocking catheter is 0.7.

In the second preferred example of Embodiment 3, the inner tube 100 includes a triple-layered structure including a first layer 101, a second layer 102 and a third layer 103 that are arranged from inside to outside. The first layer 101 has a thickness of 0.001 inch. The second layer 102 has a braided structure made from filaments with a diameter of 0.0015 inch. The third layer 103 has a thickness of 0.002 inch. The outer tube 200 includes a single-layered structure with a thickness of 0.002 inch. The flow blocking member 300 is disposed between the inner tube 100 and the outer tube 200 and includes a support frame and a covering membrane. The support frame is a cut tube with a wall thickness of 0.003 inch. The covering membrane is disposed outside the support frame and has a thickness of 0.002 inch. An outer diameter of the whole flow blocking catheter (i.e., the outer diameter of the outer tube 200) is 0.112 inch, and the inner diameter of the inner tube 100 is 0.085 inch. Therefore, the ratio of inner to outer diameter of the entire flow blocking catheter is 0.76.

In the third preferred example of Embodiment 3, the inner tube 100 includes a triple-layered structure including a first layer 101, a second layer 102 and a third layer 103 that are arranged from inside to outside. The first layer 101 has a thickness of 0.001 inch. The second layer 102 has a braided structure made from filaments with a diameter of 0.001 inch. The third layer 103 has a thickness of 0.002 inch. The outer tube 200 includes a single-layered structure with a thickness of 0.002 inch. The flow blocking member 300 is disposed between the inner tube 100 and the outer tube 200 and includes a support frame and a covering membrane. The support frame has a braided structure made from the filaments with a diameter of 0.0014 inch. The covering membrane is attached to meshes of the support frame by the dipping and drawing method and has a thickness of 0.002 inch. An outer diameter of the whole flow blocking catheter (i.e., the outer diameter of the outer tube 200) is 0.1076 inch, and the inner diameter of the inner tube 100 is 0.087 inch. Therefore, the ratio of inner to outer diameter of the entire flow blocking catheter is 0.81.

In the fourth preferred example of Embodiment 3, the inner tube 100 includes a triple-layered structure including a first layer 101, a second layer 102 and a third layer 103 that are arranged from inside to outside. The first layer 101 has a thickness of 0.0005 inch. The second layer 102 has a spiral structure made from filaments with a diameter of 0.001 inch. The third layer 103 has a thickness of 0.002 inch. The outer tube 200 includes a single-layered structure with a thickness of 0.002 inch. The flow blocking member 300 is disposed between the inner tube 100 and the outer tube 200 and includes a support frame and a covering membrane. The support frame has a braided structure made from the filaments with a diameter of 0.001 inch. The covering membrane is attached to meshes of the support frame by the dipping and drawing method and has a thickness of 0.002 inch. An outer diameter of the whole flow blocking catheter (i.e., the outer diameter of the outer tube 200) is 0.105 inch, and the inner diameter of the inner tube 100 is 0.089 inch. Therefore, the ratio of inner to outer diameter of the entire flow blocking catheter is 0.85.

The catheters of the above four preferred examples each are able to achieve good effects in use. Moreover, since the ratio of inner to outer diameter for each catheter is greater than 0.7, it allows to significantly increase the inner diameter at a limited outer diameter of the flow blocking catheter, so as to be applicable for the large-size blood clots or instruments.

### Embodiment 4

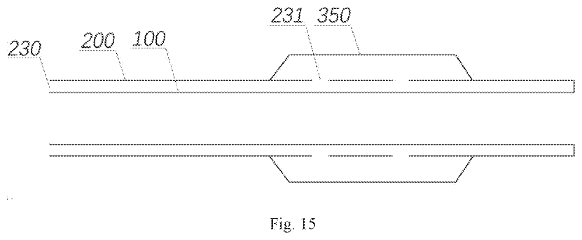

Embodiment 4 of present application provides a balloon catheter that includes an inner tube 100, a flow blocking member 300 and an outer tube 200. Referring to FIG. 15, the inner tube 100 in the balloon catheter has a triple-layered structure including a first layer, a second layer and a third layer that are arranged from inside to outside. The first layer has a thickness of 0.0005 inch. The second layer has a spiral structure made from filaments with a diameter of 0.001 inch. The third layer has a thickness of 0.002 inch. The outer tube 200 includes a single-layered structure with a wall thickness of 0.0025 inch. In this Embodiment, the flow blocking member 300 is a balloon 350 provided on the exterior of the outer tube 200. That is, each of the proximal and distal ends of the balloon 350 is coupled to the outer surface of the outer catheter 200 and a chamber is formed between the balloon 350 and the outer catheter 200. Such chamber is in fluid communication with the chamber delimited by the inner 100 and outer 200 tubes. In particular, a fluid introduction chamber 230 is delimited between the inner 100 and outer 200 tubes for introducing the liquid or gas from a proximal end of the balloon catheter to a distal end of the balloon catheter. Moreover, several fluid introduction holes are provided on the section of the outer tube 200 surrounded by the balloon 350 in order to introduce liquid or gas from the fluid introduction chamber 230 into the balloon 350 to dilate the balloon 350. In this embodiment, the balloon 350 is formed of a macromolecular material through a blow molding or extrusion approach, and has its both ends fixedly secured to the external surface of the outer tube 200 by adhesive bonding, hot pressing or welding (e.g., high frequency welding). Moreover, the balloon 350 may be collapsed via folding or other processes to facilitate its passage through blood vessels. Optionally, the balloon 350 has a wall thickness of 0.004 inch. Additionally, the flow blocking catheter has an outer diameter of 0.108 inch overall, and an inner diameter of the inner tube 100 is 0.087 inch. Therefore, the ratio of inner to outer diameter of the entire flow blocking catheter is 0.81. The balloon catheter achieves expansion of the flow blocking member 300 by introducing fluid into fluid introduction chamber, and achieves collapse of the flow blocking member 300 by drawing out the fluid. In this embodiment, the fluid is a radiopaque fluid that facilitates the operator to clearly observe the positions and states of the balloon 350 during operations of the balloon catheter.

It should be noted that the embodiments disclosed herein above are described in a progressive manner and each embodiment focuses on the differences from other embodiments. Reference can be made between the embodiments for the same or similar parts. In addition, different parts in various embodiments may be combined together, and present application is not limited to any particular combination.

In summary, the flow blocking catheter provided in present application has a large ratio of inner to outer diameter, which enables to increase the inner diameter of the flow blocking catheter at a limited outer diameter and thus makes it applicable for large-size blood clots or instruments.

The description presented above is merely a few preferred embodiments of the present application and does not limit the protection scope of present application in any sense. Any change and modification made by those of ordinary skill in the art based on the above teachings fall within the protection scope of the appended claims.

What is claimed is:

1. A flow blocking catheter, comprising:
an inner tube;
an outer tube sleeved on the exterior of the inner tube; and
a flow blocking member having at least one end coupled to an outer circumference of the inner tube or the outer tube, the flow blocking member configured to have a collasped configuration and an expanded configuration,
wherein a ratio of an inner diameter of the inner tube to an outer diameter of the outer tube is equal to or greater than 0.7.

2. The flow blocking catheter of claim 1, wherein the flow blocking member is configured to switch between the expanded and collapsed configurations under a drive of an axial movement of the outer tube relative to the inner tube.

3. The flow blocking catheter of claim 2, wherein the flow blocking member is self-expandable and sleeved on an exterior of the inner tube, and at least a proximal end of the flow blocking member is fixedly coupled to an outer circumference of the inner tube, and wherein the outer tube is movably sleeved on an exterior of the inner tube to restrict expansion of the flow blocking member.

4. The flow blocking catheter of claim 2, wherein one end of the flow blocking member is coupled to the outer circumference of the inner tube and the other end of the flow blocking member is coupled to an distal end of the outer tube, wherein the flow blocking member is configured to expand as the outer tube moves toward a distal end of the inner tube and to collapse as the outer tube moves away from the distal end of the inner tube.

5. The flow blocking catheter of claim 1, wherein the ratio of the inner diameter of the inner tube to the outer diameter of the outer tube is equal to or greater than 0.76.

6. The flow blocking catheter of claim 5, wherein the ratio of the inner diameter of the inner tube to the outer diameter of the outer tube is equal to or greater than 0.81.

7. The flow blocking catheter of claim 6, wherein the ratio of the inner diameter of the inner tube to the outer diameter of the outer tube is equal to or greater than 0.85.

8. The flow blocking catheter of claim 1, wherein the inner diameter of the inner tube ranges from 0.059 inch to 0.118 inch.

9. The flow blocking catheter of claim 1, wherein the outer diameter of the outer tube ranges from 0.078 inch to 0.137 inch.

10. The flow blocking catheter of claim 1, wherein both or either of the inner tube and the outer tube is a single-layered tube made of macromolecular material; or wherein both or either of the inner tube and the outer tube has a structure comprising at least two layers, in which both or either of a first layer and a second layer from inside to outside is a macromolecular layer; or wherein both or either of the inner tube and the outer tube has a structure comprising at least two layers, in which a second layer from inside to outside comprises one or more selected from the group consisting of braided structure, coil, and cut hypotube.

11. The flow blocking catheter of claim 1, wherein the flow blocking member comprises at least one selected from the group consisting of mesh structure, open-loop structure, spiral structure and a balloon, and wherein the flow blocking member is fabricated by braiding, winding, cutting, blow molding or extrusion.

12. The flow blocking catheter of claim 11, wherein the mesh structure is braided from 1 to 64 filaments, wherein the filament is at least one selected from the group consisting of regular filament, radiopaque filament and composite filament, the regular filament made of at least one selected from the group consisting of nickel-titanium alloy, cobalt-chromium alloy, stainless steel and macromolecular material, the radiopaque filament made of at least one selected from the group consisting of radiopaque metal, alloy of radiopaque metals and macromolecular material containing a radiopaque agent, the composite filament formed by a radiopaque core filament combined with a regular filament.
